(19) United States
(12) Patent Application Publication
(10) Pub. No.: US 2023/0257319 A1
(43) Pub. Date: Aug. 17, 2023

(54) METHODS TO PRODUCE PRODUCTS FROM ANAEROBIC DIGESTION OF POULTRY LITTER

(71) Applicants: SHAWN FREITAS, MILWAUKIE, OR (US); WILLIAM HOLLOMAN, MILWAUKIE, OR (US); BRELAND OSCAR, HILSBORO, OR (US); ALAN GILBERT, LIXNAW (IE); THOMAS M. SPANGLER, III, ANNAPOLIS, MD (US); DONAL JAMES BUCKLEY, SKIBBEREEN (IE); JOHN WALL, INNISHANNON (IE); JASON LEVINE, POTOMAC, MD (US)

(72) Inventors: SHAWN FREITAS, MILWAUKIE, OR (US); WILLIAM HOLLOMAN, MILWAUKIE, OR (US); BRELAND OSCAR, HILSBORO, OR (US); ALAN GILBERT, LIXNAW (IE); THOMAS M. SPANGLER, III, ANNAPOLIS, MD (US); DONAL JAMES BUCKLEY, SKIBBEREEN (IE); JOHN WALL, INNISHANNON (IE); JASON LEVINE, POTOMAC, MD (US)

(21) Appl. No.: 18/005,506

(22) PCT Filed: Jul. 16, 2021
(86) PCT No.: PCT/US2021/041927

§ 371 Date: Jan. 13, 2023

Related U.S. Application Data
(60) Provisional application No. 63/052,761, filed on Jul. 16, 2020.

(51) Int. Cl. C05F 17/40; B09B 3/45; C05F 3/00

(52) U.S. Cl. CPC C05F 17/40; B09B 3/45; C05F 3/00

(57) ABSTRACT

Some embodiments are directed to a process for forming products from animal waste (e.g., poultry litter). The products may include a controlled release fertilizer and renewable natural gas. The process may include providing a feedstock comprising greater than about 80 percent poultry waste and a moisture content of about 25 percent. The feedstock may be diluted to form a slurry having a moisture content of about 90%. The slurry may be mechanically refined to reduce the particle size distribution and the average particle size of the slurry. The slurry may be anaerobically digested to produce biogas and a digestate. The biogas may be converted to renewable natural gas, and the digestate may be converted to a controlled release fertilizer. Additionally, some embodiments are directed to methods of reducing nitrogen emission from soil amendments. Some embodiments are directed to reducing nitrogen emissions sufficient to produce emissions offset credits.

### CROSS-REFERENCE

This application claims the benefit of U.S. Provisional Application No. 63/052,761, filed Jul. 16, 2020, which is incorporated herein in its entirety by reference thereto.

### FIELD OF THE DISCLOSURE

This disclosure relates to methods of producing valuable products from anaerobic digestion of animal waste. More particularly, the disclosure relates to anaerobic digestion of animal waste, such as poultry litter, to produce controlled release fertilizers and renewable natural gas. Further, the methods disclosed herein can produce fertilizers that reduce nitrogen emissions.

### BACKGROUND OF THE DISCLOSURE

Anaerobic digestion systems may be used to process many types of organic matter, including, for example, animal waste. Anaerobic digestion of animal waste may be useful to produce various products, including gases and fertilizers. But existing systems oftentimes produce a large amount of waste, including wastewater and other organic matter.

Attempts have been made to use recycle streams and zero-liquid discharge systems in anaerobic digestion, but existing methods may recycle water that contains contaminants or chemical compounds that can affect the performance of the anaerobic digester. For example, some recycle streams may recycle water with nitrogen or sulfur remaining in the stream. This can create elevated levels of nitrogen and sulfur in the anaerobic digester. Eventually this will cause the nitrogen and sulfur levels to build up in the digester, which is nearly always detrimental to digester performance.

Issues with nitrogen and sulfur in the recycle stream are compounded when the feedstock already contains high concentrations of nitrogen and sulfur. For example, animal wastes typically have higher nitrogen levels that other organic matter that may be anaerobically digested. And within animal waste, poultry waste contains the highest levels of nitrogen. Accordingly, there is a need for a system that can accept high-nitrogen feedstock, includes a recycle stream, and discharges little to no liquid from the system.

Using processes according to embodiments disclosed herein, it is possible to use anaerobic digestion to produce value-added products (e.g., fertilizer and biogas) from animal waste (e.g., poultry litter).

### BRIEF SUMMARY OF THE DISCLOSURE

Some embodiments are directed to a process for forming a renewable natural gas, the process comprising providing a feedstock, the feedstock comprising greater than 80 percent poultry waste, the feedstock having a moisture content of about 35 percent or less; diluting the feedstock to form a slurry having a moisture content of at least about 80%; mechanically refining the slurry such that the slurry has an average particle size of about 100 μm or less; anaerobically digesting the slurry to produce a digestate having a solids content of less than about 10% by weight and a biogas; removing carbon dioxide from the biogas to produce a renewable natural gas having a methane concentration of at least 70% by volume.

In any of the various embodiments disclosed herein, the feedstock has a moisture content of about 10% to about 50%.

In any of the various embodiments disclosed herein, the slurry has a moisture content of about 90% to about 98%.

In any of the various embodiments disclosed herein, the digestate has a solids content of about 2% to about 7%. In any of the various embodiments disclosed herein, the digestate has a solids content of about 3.5%.

In any of the various embodiments disclosed herein, the slurry has an average particle size of about 10 μm or less.

In any of the various embodiments disclosed herein, the renewable natural gas has a methane concentration from about 40% to about 90%.

In any of the various embodiments disclosed herein, the mechanical refining is done before the anaerobic digestion.

In any of the various embodiments disclosed herein, the mechanical refining is done using a conical refiner.

In any of the various embodiments disclosed herein, the mechanical refining is done using a disc refiner.

In any of the various embodiments disclosed herein, the feedstock comprises greater than 90 percent poultry waste.

In any of the various embodiments disclosed herein, the process further comprises removing impurities from the biogas, wherein the impurities comprise at least one of ammonia or hydrogen sulfide.

In any of the various embodiments disclosed herein, the impurities are removed before the carbon dioxide is removed.

In any of the various embodiments disclosed herein, the renewable natural gas is produced at a rate of at least about 2000 MMBTU per day.

In any of the various embodiments disclosed herein, the process further comprises producing a controlled release fertilizer using the digestate.

In any of the various embodiments disclosed herein, the biogas comprises methane, and the process further comprises reacting at least a portion of the methane with steam at a pressure from about 3 bar to about 15 bar to produce hydrogen.

In any of the various embodiments disclosed herein, the process further comprises separating the biogas to produce an acid-rich stream and producing a controlled release fertilizer using the acid-rich stream and the digestate.

In any of the various embodiments disclosed herein, the process further comprises separating water from the digestate for recycle.

Some embodiments are directed to a process of preparing a controlled release fertilizer composition comprising anaerobically digesting poultry litter to produce a digestate having a solid fraction and a liquid fraction, the digestate comprising at least about 1500 mg/L of combined tannins, humins and fulvins.

In any of the various embodiments disclosed herein, the process further comprises adding an acid to the solid fraction to produce a humates sludge paste.

In any of the various embodiments disclosed herein, the acid is sulfuric acid.

In any of the various embodiments disclosed herein, the adding the acid reduces the pH to about 2 to about 4.

In any of the various embodiments disclosed herein, the process further comprises adding a base to the solid fraction to produce a fulvates sludge paste.

In any of the various embodiments disclosed herein, the base is potassium hydroxide.

In any of the various embodiments disclosed herein, the adding the base increases the pH to about 7 to about 9.

In any of the various embodiments disclosed herein, the process further comprises treating the liquid fraction to produce brine solids.

In any of the various embodiments disclosed herein, the brine solids are polyhalite brine solids.

Some embodiments are directed to a controlled release fertilizer composition comprising tannins, humins, fulvins, and brine solids, wherein the controlled release fertilizer is derived from a poultry litter digestate, wherein the combined composition of tannins, humins, and fulvins is at least about 1500 mg/L.

In any of the various embodiments disclosed herein, the composition further comprises one or more silicates.

In any of the various embodiments disclosed herein, wherein the one or more silicates comprise clay and zeolite species.

In any of the various embodiments disclosed herein, wherein the composition comprises about 1% by weight to about 20% by weight silicates.

In any of the various embodiments disclosed herein, the clay and zeolite species comprise halloysite, kaolinite, illite, montmorillonite, talc, sepiolite, pyrophyllite, erionite, stilbite, mordenite, or combinations thereof.

In any of the various embodiments disclosed herein, the nitrogen absorbed by the controlled release fertilizer is released from the controlled release fertilizer at the rate of less than or equal to about 15% in 24 hours or less than or equal to about 75% in 28 days.

Some embodiments are directed to a method of reducing nitrogen emissions by applying a composition comprising a controlled release fertilizer as a soil amendment.

In any of the various embodiments disclosed herein, the nitrogen emissions may be reduced by at least about 10%. In any of the various embodiments disclosed herein, the nitrogen emissions may be reduced by at least about 25%.

### BRIEF DESCRIPTION OF THE FIGURES

FIG. 1 shows a flow chart of an exemplary process for treating animal waste using anaerobic digestion.

FIG. 2 shows a flow chart of an exemplary process for treating an animal waste feedstock.

FIG. 3 shows a flow chart of an exemplary process for digesting an animal waste feedstock.

FIG. 4 shows a flow chart of an exemplary process for converting biogas to renewable natural gas.

FIG. 5 shows a flow chart of an exemplary process for using renewable natural gas generated by the process shown in FIG. 4.

FIG. 6 shows a flow chart of an exemplary process for converting digestate to brine solids.

FIG. 7 shows a flow chart of an exemplary process for converting digestate to sludge pastes.

FIG. 8 shows a chart of various process parameters over time.

FIG. 9 shows a chart of process water turbidity and conductivity over time.

### DETAILED DESCRIPTION OF THE DISCLOSURE

The present disclosure is directed to a process for converting animal waste (e.g., poultry litter) into valuable products, including biogas and a controlled-release fertilizer through anaerobic digestion. The biogas can be processed to produce end products, such as renewable natural gas. The digestate can be processed to produce end products, such as controlled-release fertilizer. The system can be a closed loop system. Additionally, the system and the products can be processed to achieve a reduction in nitrogen emissions.

Before the present disclosure is described in greater detail, it is to be understood that this disclosure is not limited to the particular compositions or process steps described, which can, of course, vary. As will be apparent to those of skill in the art upon reading this disclosure, each of the individual aspects described and illustrated herein has discrete components and features which can be readily separated from or combined with the features of any of the other several aspects without departing from the scope or spirit of the present disclosure. Any recited method can be carried out in the order of events recited or in any other order which is logically possible.

The headings provided herein are not limitations of the various aspects of the disclosure, which can be defined by reference to the specification as a whole. It is also to be understood that the terminology used herein is for the purpose of describing particular aspects only, and is not intended to be limiting, since the scope of the present disclosure will be limited only by the appended claims.

### Terms

In order that the present disclosure can be more readily understood, certain terms are first defined. As used in this application, except as otherwise expressly provided herein, each of the following terms shall have the meaning set forth below. Additional definitions are set forth throughout the application.

It is to be noted that the term “a” or “an” entity refers to one or more of that entity. As such, the terms “a” (or “an”), “one or more,” and “at least one” can be used interchangeably herein. It is further noted that the claims can be drafted to exclude any optional element. As such, this statement is intended to serve as antecedent basis for use of such exclusive terminology as “solely,” “only” and the like in connection with the recitation of claim elements, or use of a negative limitation.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Numeric ranges are inclusive of the numbers defining the range. Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither or both limits are included is also encompassed within the disclosure. Thus, ranges recited herein are understood to be shorthand for all of the values within the range, inclusive of the recited endpoints. For example, a range of 1 to 10 is understood to include any number, combination of numbers, or sub-range from the group consisting of 1, 2, 3, 4, 5, 6, 7, 8, 9, and 10.

Where a value is explicitly recited, it is to be understood that values which are about the same quantity or amount as the recited value are also within the scope of the disclosure. Where a combination is disclosed, each sub-combination of the elements of that combination is also specifically disclosed and is within the scope of the disclosure. Conversely, where different elements or groups of elements are individually disclosed, combinations thereof are also disclosed. Where any element of a disclosure is disclosed as having a plurality of alternatives, examples of that disclosure in which each alternative is excluded singly or in any combination with the other alternatives are also hereby disclosed; more than one element of a disclosure can have such exclusions, and all combinations of elements having such exclusions are hereby disclosed.

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower).

The term “fertilizer” is used herein to include a fertilizer, a soil conditioner, and/or a biostimulant.

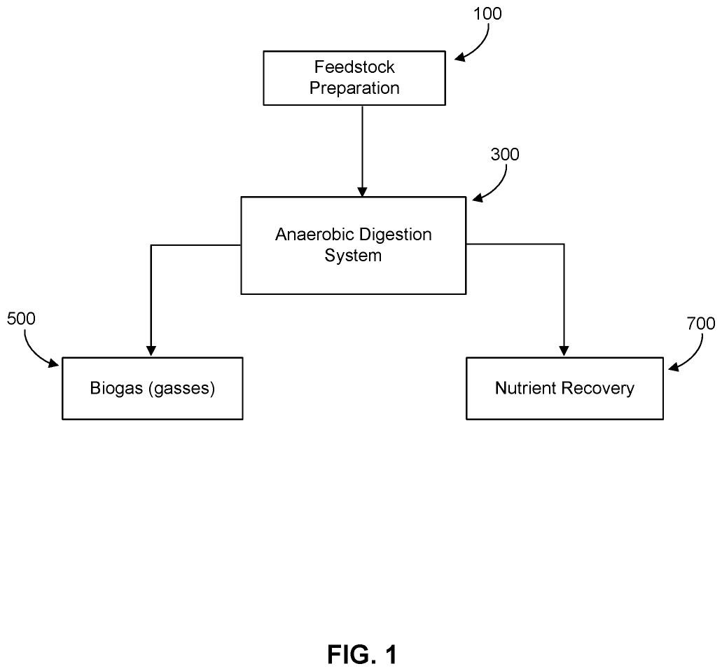

As illustrated in FIG. 1 and discussed in more detail, processes disclosed herein include methods and systems for producing products from animal waste (e.g., poultry litter). The methods and processes may include feedstock preparation 100, anaerobic digestion 300, biogas conversion 500, and nutrient recovery 700. Each of these is discussed in more detail below.

### Feedstock Preparation

In one aspect, the feedstock is poultry waste. Poultry waste for purposes of the present disclosure means any waste from poultry operations, including broiler operations and layer operations. Poultry may mean any kind of bird kept for the production of eggs, meat or feathers. The poultry waste can come from poultry of any age, from broiler operations or layer operations. Such waste can include: solid or liquid waste generated from the animals; biomass, such as litter from the animal beds, which includes wood shavings, saw dust, straw, peanut hulls, or others absorbent materials; spilled food; animal feathers; and dead animals. Poultry waste typically comprises a combination of these types of waste.

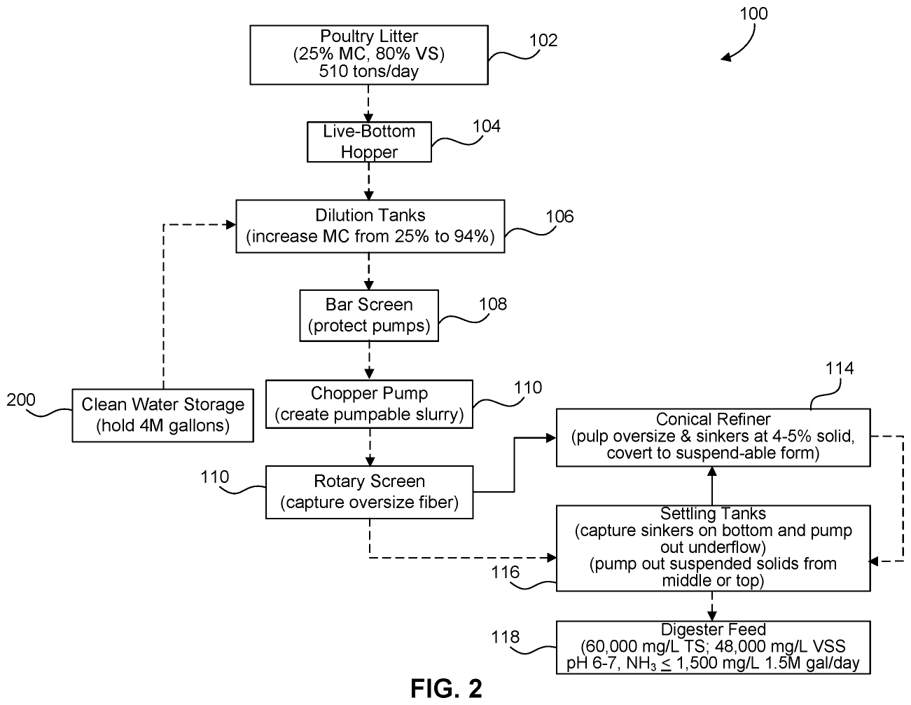

FIG. 2 illustrates an exemplary feedstock preparation process 100 for preparing a feedstock (e.g., feedstock 102). As illustrated in FIG. 2, feedstock preparation 100 may include feeder 104, dilution tanks 106, screen 108, pump 110, separator 112, mechanical refiner 114, settling tanks 116, and digester feed 118.

Feedstock 102 may be provided to the system using feeder 104. The feedstock for the processes describe herein may include at least 50% poultry waste (e.g., at least 60% poultry waste, at least 70% poultry waste, at least 80% poultry waste, at least 90% poultry waste, or at least 95%). In some embodiments, the poultry waste has a moisture content of about 10% to about 50% (e.g., about 20% to about 40% or about 20% to about 30%). In some embodiments, the poultry waste has a moisture content of about 35% or less (e.g., about 30% or less, about 25% or less, about 20% or less, about 15% or less, about 10% or less). In some embodiments, the poultry waste has a moisture content of about 25%. In some embodiments, the poultry waste has a moisture content of at least 50% (e.g., at least about 60%, at least about 70%, or at least about 75%). The feedstock may be provided at a rate of at least 400 tons per day (e.g., at least 500 tons per day, at least 600 tons per day, or at least 700 tons per day). In some embodiments, the feedstock is provided at a range of about 400 tons per day to about 700 tons per day (e.g., about 500 tons per day to about 600 tons per day). In some embodiments, the feedstock is provided at a rate of about 510 tons per day.

The feedstock may be transferred to the system using feeder 104. Feeder 104 may include at least one suitable mechanism for feeding or metering the feedstock. For example, feeder 104 may include at least one screw feeder or at least one live-bottom hopper. In some embodiments, feeder 104 is a live-bottom hopper.

Using feeder 104, feedstock 102 may be provided to at least 1 dilution tank (e.g., at least 2 dilution tanks or at least 3 dilution tanks). In some embodiments, feedstock preparation process 100 includes between 1 dilution tank and 6 dilution tanks (e.g., 2 dilution tanks, 3 dilution tanks, 4 dilution tanks, or 5 dilution tanks). Water from water storage 200 may be provided to the dilution tanks to increase the moisture content of feedstock 102. The moisture content of feedstock 102 may be increased to at least 70% (e.g., at least 80% or at least 90%, at least 92%, at least 94%, at least 96%, at least 98%, or at least 99%). In some embodiments, the moisture content of feedstock 102 is increased to about 90% to about 98%. In some embodiments, the moisture content is increased to about 94%. In some embodiments, the dilution tanks may reduce the solids content to about 5% to about 10%.

After dilution, feedstock 102 may be transferred to pump 110 to create a pumpable slurry. For example, the biomass in feedstock 102 may be reduced to a particle size distribution of about 2 inches to about 0.25 inches. In some embodiments pump 110 includes at least 2 pumps (e.g., at least 3 pumps). Pump 110 may be a chopper pump or a grinder pump. When passed through pump 110, feedstock 102 may be a pumpable slurry. As used herein, “pumpable slurry” means a slurry having a particle size distribution of between about 2 inches and about 0.25 inches. Screen 108 may be optionally installed between dilution tanks 106 and pump 110 to prevent oversized solids from entering pump 110, which could damage pump 110.

Once feedstock 102 has been formed into a pumpable slurry, the pumpable slurry may be passed through separator 112 that separates the pumpable slurry based on particle size. For example, separator 112 may separate the pumpable slurry into a first fraction that containing oversized particles (i.e., particles that are too large to transfer to the anaerobic digester) and a second fraction containing smaller particles. In some embodiments, the pumpable slurry is not passed through separator 112 and is instead transferred directly to mechanical refiner 114. For example, oversized particles may be particles having an average size of about 0.5 inches or greater. Optionally, at least 1 settling tank 116 (e.g., at least 2 settling tanks or at least 3 settling tanks) may be downstream of separator 112. The first fraction may be transferred to a mechanical refiner 114 (e.g., a conical refiner or disc refiner) for further refining before transfer to the settling tank 116. The mechanical refining is discussed in more detail below. The second fraction may be transferred to settling tank 116. Settling tank 116 may capture sinking particles on the bottom and pump out the sinking particles underflow. Settling tank 116 may pump out suspended solids from the middle or the top. The second fraction may be set in one or more holding tanks (e.g., settling tanks 116) for about 12 hours to about 36 hours to allow the pumpable slurry to soften. This allows some of the exposed lignocellulosic fibers to swell and open, but not to an extent that allows for premature microbial action on the cellulose and hemicellulose fibers. This reduces the loss of volatile solids, which in turn reduces energy consumption across the refiner and maximizes cleavage impacts and fiber damage. Further, this setting step serves as a feed reservoir to support steady state operation of the anaerobic digesters regardless of interruptions related to feedstock delivery rates. These holding tanks (e.g., settling tanks 116) may operate at elevated temperatures (e.g., from about 70° F. to about 100° F.) which improves the softening and opening of the lignocellulosic matrix. The water in the holding tanks may be sourced from downstream digesters (e.g., digesters 304 and 306), and such water may include appropriate microbes such that the ecosystem continuity is maintained.

Following mechanical refining, discussed in more detail below, other methods may be used to increase the maximum levels of cellulose and hemicellulose are made available to microbial attack, breakdown, and ultimately conversion into biogas. For example, nanocellulose production methods may be used to reduce the molecular weight of the cellulose fragments and further accelerate microbial decomposition. This may be done using high pressure water jets. By colliding two high pressure slurry jets at an angle (e.g., at an oblique angle) or by colliding a single high-pressure slurry jet with something impermeable can further reduce particle size. As particle size is reduced, the rate of microbial degradation increases.

### Mechanical Refining

A mechanical refiner 114 may be used upstream of the digester (e.g., digesters 304 and 306) to further reduce the particle size of solids in the feedstock. This particle size reduction serves to maximize the levels of polymers that are beneficial for producing a high-quality controlled release fertilizer. For example, mechanical refiner 114 may reduce particles sizes to increase the levels of produced polyelectrolytes and polyphenolics. These are highly functional and have a high molecular weight and a high charge density. In some embodiments, mechanical refiner 114 reduces particle sizes such that levels of tannins, humins, and fulvins are increased in their acid or salt forms.

Mechanical refining is beneficial for accelerating the rate of microbial biomass degradation. In some embodiments, mechanical refining takes place just before digestion to reduce the loss of volatile solids that would otherwise be converted to biogas. Generally, mechanical refining using a conical refiner is achieved by pumping the feed between a rotating grinder (i.e., a rotor) and a stationary cutter (i.e., a stator) each with radial grooves that provide a grinding/cutting surface. As the feed is pumped through the mechanical refiner, the feed is broken down as applied forces push it towards the outlet where the grooves are finer. Ultimately, the mechanical refining reduces a lignified biomass feed to a biomass fiber slurry. The size of the refined fibers can be controlled by altering the distance between the rotor and stator. For example, less distance between the rotor and stator produces finer fibers but also requires higher grinding force. The distance between the rotor and stator can also affect the chemical oxygen demand (“COD”) of the slurry, as shown in Example 2 below.

Mechanical refiner 114 may be a conical refiner, disc refiner, plate refiner, colloid mill, or similar mechanical refiner. In some embodiments, mechanical refiner 114 is a conical refiner. In some embodiments, mechanical refiner 114 is a disc refiner. Mechanical grinders used in wastewater treatment plants, pulp and paper processes, etc. typically only reduce particle size to 0.25 inches. Reduction beyond 0.25 inches requires more expensive equipment that makes further size reduction economically impractical. And the levels of hydrolysis and oxidative breakdown in existing systems make reduction below 0.25 inches unnecessary. For example, reducing particle size to 0.25 inches can typically be done with grinder and choppers using cutter heads with teeth and blades, but refiners or mills are typically required for further size reduction. Mechanical refining according to embodiments disclosed herein are different than mechanical grinding/chopping used in, for example, wastewater processing or pulp and paper application. For example, the specific objective of pulp and paper processes is to recover functional fiber from a lignified biomass chip. Such functional fiber must have sufficient length and strength to be useful in paper products. Accordingly, a pulp and paper application would not include mechanical grinding upstream of the refiner. Smaller particles sizes yield weaker pulps that contain more damaged fibers and higher levels of extractives (e.g., higher chemical oxygen demand (“COD”), higher biological oxygen demand (“BOD”), and higher volatile suspended solids (“VSS”). This results in a slurry with undesirable characteristics for the production of paper products.

In contrast, according to embodiments disclosed herein, the mechanical refining may be applied to thinner and/or smaller chips, which means energy is absorbed differently than thicker and bulkier chips (i.e., such as those utilized during pulp and paper processes). As disclosed herein, thinner and smaller chips result in more cleavage failure and less forward shear compared to thicker and bulkier chips. Accordingly, the energy applied is more plastic (i.e., less elastic), and more energy is applied to bond-breaking, thus creating larger amounts of surface area per impact. These cleavage impacts are severe and cause fiber damage and shattering which generates pulps with more broken and less fibrillated fibers. Maximizing the level of cleavage failure across the mechanical refiner allows for improved setting and swelling compared to lower levels of cleavage. For example, the level of cleavage may be maximized so as to deliberately initiate hydrolysis and weaken the fiber. This helps to reduce energy consumption while also increasing the production of volatile solids that can be converted to biogas.

Mechanical refiner 114 may reduce particles sizes such that the particle size distribution in the slurry is from about 3500 μm to about 1 μm (e.g., from about 2500 μm to about 1 μm, from about 1500 μm to about 1 μm, from about 500 μm to about 1 μm, from about 100 μm to about 1 μm, from about 50 μm to about 1 μm, or from about 30 μm to about 1 μm). In some embodiments, the particle size distribution is from about 30 μm to about 1 μm. Mechanical refiner 114 may reduce particle sizes to an average particle size of about 100 μm or less (e.g., about 75 μm or less, about 50 μm or less, about 30 μm or less, about 20 μm or less, or about 10 μm or less) in the slurry. In some embodiments, the average particle size of the slurry is about 1 μm to about 1000 μm (e.g., about 10 μm to about 1000 μm or about 1 μm to about 100 μm).

Once feedstock 102 has reached mechanical refiner 114, it may be a biomass-rich slurry. The inventors have unexpectedly found that production of valuable products such as biogas (e.g., renewable natural gas) and controlled release fertilizers may be maximized by further processing the biomass-rich slurry using a mechanical refiner (e.g., a conical refiner or a disc refiner) to breakdown the biomass, expose the cellulose and hemicellulose, and produce a slurry rich in fiber and soluble organics. For example, compared to systems that do not use the additional mechanical refining, biogas production may be increased by at least about 10% (e.g., at least 20%, at least 30%, or at least 40%). In some embodiments, biogas conversion is increased by about 20% to about 30%. Without being bound by theories, it is believed that further processing increases the relative concentration of lignin degradation products (e.g., tannic, humic, or fulvic acids) in the digestate.

Lignin is a heterogeneous polymer that lacks a defined primary structure. It is a cross-linked polymer with typical molecular masses in excess of 10,000 u, and is relatively hydrophobic and rich in aromatic subunits (lignols). Some lignols will crosslink, including all derived from phenylpropane, all of which are methoxylated to various degrees: p-coumaryl alcohol, coniferyl alcohol, and sinapyl alcohol. These lignols are incorporated into lignin in the form of the phenylpropanoids p-hydroxyphenyl (H), guaiacyl (G), and syringyl (S), respectively. The lignin degradation products (e.g., tannic-like, humic, and fulvic acids) have exceptionally high ion-loading capacities, which allows them to act as ion-exchange buffers for free ions in solution. This slows transfer and reactivity, which in turn enables the digesters (e.g., digesters 304 and 306) to operate at high salt levels (NH₃, K, Na, Ca, Mg, etc.) than would be otherwise possible.

Another unexpected benefit to using mechanical refiner 114 (e.g., conical refiner or disc refiner) is the effect on the downstream processing of the digestate to recover nutrients in a controlled, slow-release form. As discussed above, the digestate may have a particle size distribution from about 100 μm to about 1 μm (e.g., about 30 μm to about 1 μm) and an average particle size of about 10 μm or less, which means the digestate has particle sizes in a similar range as silt. A digestate with these characteristics can produce, using anaerobic digestion, a solids fraction that be processed using drying and compounding methods typically only useful on silt and clay minerals. It has been unexpectedly found that such processing reduces the costs of producing controlled, slow-release fertilizer (the controlled, slow-release fertilizer is discussed in more detail below). For example, a smaller particle allows greater control over homogeneity of the end-product (e.g., controlled release fertilizer) from both a composition and morphology perspective. Smaller particles sizes allow blending a repeatable target composition and then repeatable re-form into the target morphology. This control over homogeneity is generally not possible with large particles.

Another unexpected benefit to using mechanical refiner 114 (e.g., conical refiner or disc refiner) is increased exposure of carbon during mechanical refining, which has numerous benefits to the process. Without being bound by theories, it is believed that mechanical refining as disclosed herein exposes more carbon in the resulting slurry, which can improve the balance of carbon and protein and improve the ratio of carbon, hydrogen, and nitrogen within the digester. This can in turn reduce ammonia levels, which decreases the possibility of ammonia toxicity within the digester and increases biogas production.

### Anaerobic Digestion

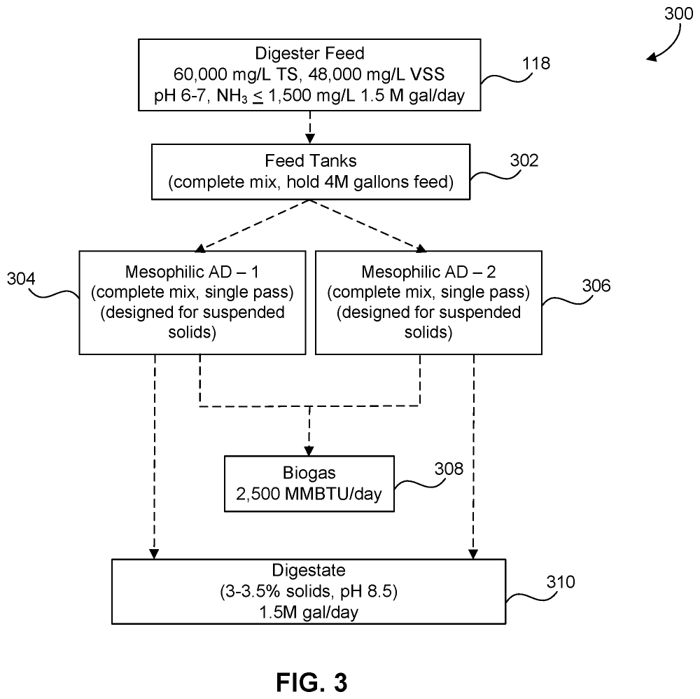

As illustrated in FIG. 3, the anaerobic digestion process may include digester feed 118 from feedstock preparation 100, feed tanks 302, and one or more digesters (e.g., digesters 304 and 306). In some embodiments, at least 2 digesters (e.g., at least 4 digesters) may be used. In some embodiments, 1 or more digesters are used (e.g., 1 digester, 2 digesters, 3 digesters, 4 digesters, 5 digesters, 6 digesters, 7 digesters, 8 digesters, 9 digesters, or 10 digesters). The anaerobic digestion process illustrated in FIG. 3 may produce biogas 308 and digestate 310.

The anaerobic digestion input produced by the feedstock preparation described above may be metabolizable carbohydrate and low molecular weight organic chemicals. The anaerobic digestion system may use anaerobic microorganisms (e.g., acetoclasts or hydrogenotrophs) to consume the input and produce biogas and a digestate liquid. As discussed in more detail below, the biogas may include methane (CH₄), carbon dioxide (CO₂), water vapor, and various impurities (e.g., ammonia (NH₃) and hydrogen sulfide (H₂S)). The biogas may ultimately get upgraded to a renewable natural gas, as discussed below. The digestate may include liquids and solids that may be converted to valuable products, such as a controlled, slow-release fertilizer.

The mechanically refined slurry may exit the one or more holding tanks (e.g., settling tanks 116) as digester feed 118. Digester feed 118 may be transferred to feed tanks 302. Digester feed 118 may be transferred to feed tanks 302 at a rate of at least 500,000 gallons per day (e.g., at least 1 million gallons per day, at least 1.5 million gallons per day, or at least 2 million gallons per day). In some embodiments, digester feed 118 may be transferred to feed tanks 302 at a rate of about 500,000 gallons to about 3 million gallons per day (e.g., about 1 million gallons per day to about 2 million gallons per day). Digester feed 118 may include a total solids concentration from about 40,000 mg/L to about 100,000 mg/L (e.g., from about 50,000 mg/L to about 90,000 mg/L, from about 50,000 mg/L to about 80,000 mg/L, from about 50,000 mg/L to about 70,000 mg/L). In some embodiments, the total solids concentration is about 60,000 mg/L. Digester feed 118 may include ammonia levels of less than 2000 mg/L (e.g., less than 1500 mg/L or less than 1000 mg/L). In some embodiments, digester feed 118 may include a VSS concentration from about 35,000 mg/L to about 65,000 mg/L (e.g., from about 45,000 mg/L to about 55,000 mg/L). In some embodiments, digester feed 118 has a VSS concentration of about 48,000 mg/L. Digester feed may have a pH of about 5 to about 8 (e.g., about 6 to about 7).

Digester feed 118 may be transferred to one or more feed tanks 302. Feed tanks 302 may have a total capacity of at least 2 million gallons (e.g., at least 3 million gallons, at least 4 million gallons, or at least 5 million gallons). In some embodiments, feed tanks 302 may have a total capacity of about 2 million gallons to about 5 million gallons (e.g., about 3 million gallons to about 4 million gallons). In some embodiments, feed tanks 302 have a capacity of about 4 million gallons.

From feed tanks 302, the digester input may be transferred at a rate sufficient to maintain steady state operation of the one or more anaerobic digesters (e.g., digesters 304 and 306). Each anaerobic digester (e.g., digesters 304 and 306) may operate in mesophilic conditions, thermophilic conditions, or psychrophilic conditions The anaerobic digesters may operate at mesophilic conditions at temperatures from about 20° C. (68° F.) to about 45° C. (113° F.) (e.g., from about 25° C. (77° F.) to about 40° C. (104° F.)). In some embodiments, the anaerobic digesters 304 and 306 operate at about 37° C. (98.6° F.). The anaerobic digesters may operate at thermophilic conditions at temperatures from about 40° C. (104° F.) to about 120° C. (248° F.) (e.g., from about 40° C. (104° F.) to about 100° C. (212° F.), from about 50° C. (122° F.) to about 80° C. (176° F.), or from about 50° C. (122° F.) to about 60° C. (140° F.)). The anaerobic digesters may operate at psychrophilic conditions at temperatures from about −20° C. (−4° F.) to about 20° C. (68° F.) (e.g., from about −10° C. (14° F.) to about 10° C. (50° F.)).

The one or more anaerobic digesters (e.g., digesters 304 and 306) may produce biogas 308 and digestate 310. Biogas may be produced at a rate of at least 1500 MMBTU per day (e.g., at least 2000 MMBTU per day, at least 2500 MMBTU per day, at least 3000 MMBTU per day, or at least 3500 MMBTU per day). In some embodiments, biogas may be produced at a rate of about 1500 MMBTU to about 5000 MMBTU (e.g., about 2000 MMBTU to about 4000 MMBTU or about 2000 MMBTU to about 2500 MMBTU). In some embodiments, biogas is produced at a rate of about 2500 MMBTU per day. Digestate 310 may be produced at a rate of at least about 500,000 gallons per day (e.g., at least about 1 million gallons per day, at least about 1.5 million gallons per day, or at least about 2 million gallons per day). In some embodiments, digestate 310 may be produced at a rate of about 500,000 gallons per day to about 3 million gallons per day (e.g., about 1 million gallons per day to about 2 million gallons per day). In some embodiments, digestate is produced at a rate of about 1.5 million gallons per day. Digestate may include a solids content of less than about 10% (e.g., less than about 7%, less than about 5% or less than about 3%). Digestate may include a solids content by weight of about 2% to about 7% (e.g., about 3% to about 6% or about 4% to about 5%). In some embodiments, the digestate has a solids content of about 3.5%. The digestate may have a pH of at least 7.5 (e.g., at least 8, at least 8.5, or at least 9). In some embodiments, digestate has a pH of about 8.5.

### Biogas Processing

The biogas produced by the digesters (e.g., digester 304 and 306) may include methane (CH₄), carbon dioxide (CO₂), water vapor, and various impurities (e.g., ammonia (NH₃) and hydrogen sulfide (H₂S)). The biogas may be further processed to produce pipeline quality renewable natural gas (“RNG”). In some embodiments, the pipeline quality RNG includes about 0.5 grains or less of total sulfur per 100 standard cubic feet and is composed of either (1) at least about 70 percent methane by volume or (2) a gross calorific value from about 950 to about 1100 Btu per standard cubic foot. In some embodiments, the pipeline quality RNG comprises at least about 80% methane by volume (e.g., at least about 90% methane by volume, at least about 95% methane by volume, or at least about 97% methane by volume). In some embodiments, the pipeline quality RNG comprises about 80% methane to about 99% methane (e.g., about 90% methane to about 97% methane). The biogas from the digesters is primarily composed of methane and carbon dioxide, but it also contains water and impurities. The biogas may have a methane to CO₂ ratio of about 1:1 to about 3:1. Pipeline quality RNG may be produced from the biogas by drying the gas and removing CO₂ and impurities.

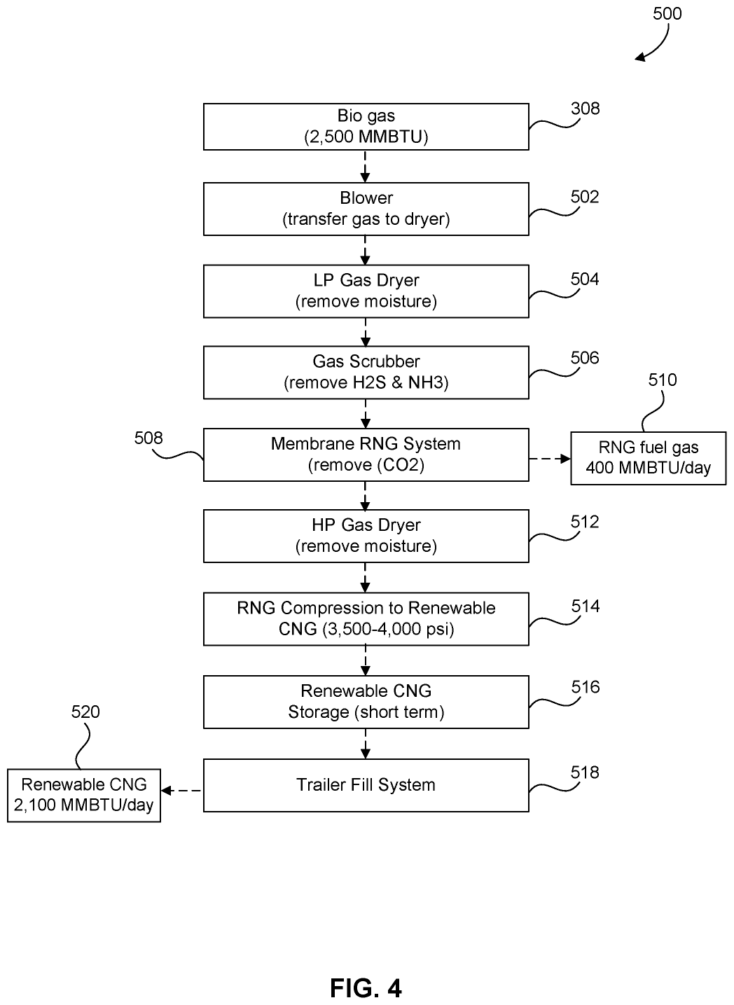

As illustrated in FIG. 4, biogas processing system 500 may include biogas 308, blower 502, first gas dryer 504, gas scrubber 506, separator 508, second gas dryer 510, compressor 512, storage 514, and fill system 516. Biogas processing system 500 may include renewable natural gas 518 that is used as fuel for the overall process and compressed RNG 520.

Biogas 308 may be transferred by blower 502 to first gas dryer 504 to remove water from the biogas. Biogas 308 will typically have a temperature approximately corresponding to the operating conditions within the digester. In some embodiments, biogas 308 has a temperature in a range of about 90° F. to about 110° F. (e.g., about 95° F. to about 100° F.). Biogas 308 may have a water content of about 2% to about 10%. In some embodiments, biogas 308 has a water content of about 5%. In some embodiments, biogas 308 is fully saturated. First gas dryer 504 may be a low pressure gas dryer. First gas dryer 504 may reduce the water content to less than about 2%. In some embodiments, first gas dryer 504 includes cooling and coalescing filters or equivalent. Water removed from the biogas may be returned to another component of the process.

After first gas dryer 504, the biogas may be transferred to gas scrubber 506 to remove at least a portion of the impurities, such as NH₃ and H₂S. Gas scrubber 506 may include adsorbents (e.g., iron oxide adsorbents) in a lead-lag bed configuration or equivalent. Gas scrubber 506 may reduce the impurities concentration to less than about 1% (e.g., less than 0.5% or less than 0.1%). In some embodiments, gas scrubber 506 may reduce the concentration of impurities (e.g., NH₃ and/or H₂S) to less than about 25 ppm (e.g., less than about 10 ppm or less than about 5 ppm). In some embodiments, gas scrubber 506 may reduce the concentration of impurities (e.g., NH₃ and/or H₂S) to less than about 50 ppb (e.g., less than about 25 ppb or less than about 10 ppb).

Gas scrubber 506 may remove CO₂ using a separator 508. In some embodiments, the CO₂ and impurities may be removed together. In some embodiments, separator 508 is a membrane separator. Separator 508 may produce a CO₂-rich stream and a CH₄-rich stream comprising at least about 40% methane by volume (e.g., at least about 50%, at least about 60%, at least about 70%, at least about 80% methane by volume, at least about 85% methane by volume, at least about 90% methane by volume, at least about 95% methane by volume, or at least about 99% methane by volume). In some embodiments, the CH₄-rich stream comprises about 40% to about 99% methane by volume (e.g., about 70% to about 95% methane by volume, or about 80% to about 90% by volume). In some embodiments, the CH₄-rich stream is pipeline quality RNG. The CO₂-rich stream may optionally be reacted with ammonia sourced from gas scrubber 506 to produce urea. The urea may be used downstream to produce various aldehydes to generate slow-release fertilizers. For example, the urea may be used to form urea-formaldehyde, urea-isobutyraldehyde/isobutylidene diurea, and/or urea-acetaldehyde/cyclo diurea.

The CH₄-rich stream may be used for one or more of fuel for the process (e.g., renewable natural gas 518), production of renewable hydrogen at reformer 519, or production of compressed RNG 520. The CH₄-rich stream may be optionally passed through second gas dryer 510. In some embodiments, second gas dryer 510 is a high pressure gas dryer. Each of first gas dryer 504 and second gas dryer 510 may operate at pressure of about 0.1 psi to about 700 psi (e.g., about 0.1 psi to about 14.7 psi, about 50 psi to about 700 psi, about 100 psi to about 600 psi, about 200 psi to about 500 psi, or about 300 psi to about 400 psi).

The RNG may be transferred to compressor 512, which may compress the RNG to pressures of about 3500 psi to about 4000 psi. In some embodiments, the RNG is compressed to a pressure of about 3600 psi. At this pressure, the RNG can be filled in standard road transport trucks that are capable of injecting the RNG into pipelines. For example, compressed RNG may be stored in storage 514 before being transported to fill system 516. Biogas processing system 500 may be capable of producing renewable natural gas to use as fuel for the system at a rate of at least about 300 MMBTU per day (e.g., at least 400 MMBTU per day, or at least 500 MMBTU per day). In some embodiments, biogas processing system 500 may be capable of producing renewable natural gas to use as fuel for the system at a rate of about 300 MMBTU to about 600 MMBTU (e.g., about 400 MMBTU to about 500 MMBTU). In some embodiments, biogas processing system 500 produces about 400 MMBTU per day of RNG that is used for fuel for the process. Further, in addition to gas used as fuel for the system, biogas processing system 500 may be capable of producing compressed RNG for offloading at fill system 516 at a rate of at least about 1500 MMBTU per day (e.g., at least 1800 MMBTU per day, at least 2100 MMBTU per day, or at least 2400 MMBTU per day. In some embodiments, biogas processing system 500 may be capable of producing compressed RNG for offloading at a rate of about 1500 MMBTU to about 2500 MMBTU (e.g., about 1800 MMBTU to about 2400 MMBTU or about 2100 MMBTU to about 2400 MMBTU). In some embodiments, biogas processing system 500 produces about 2100 MMBTU per day of compressed RNG that is offloaded.

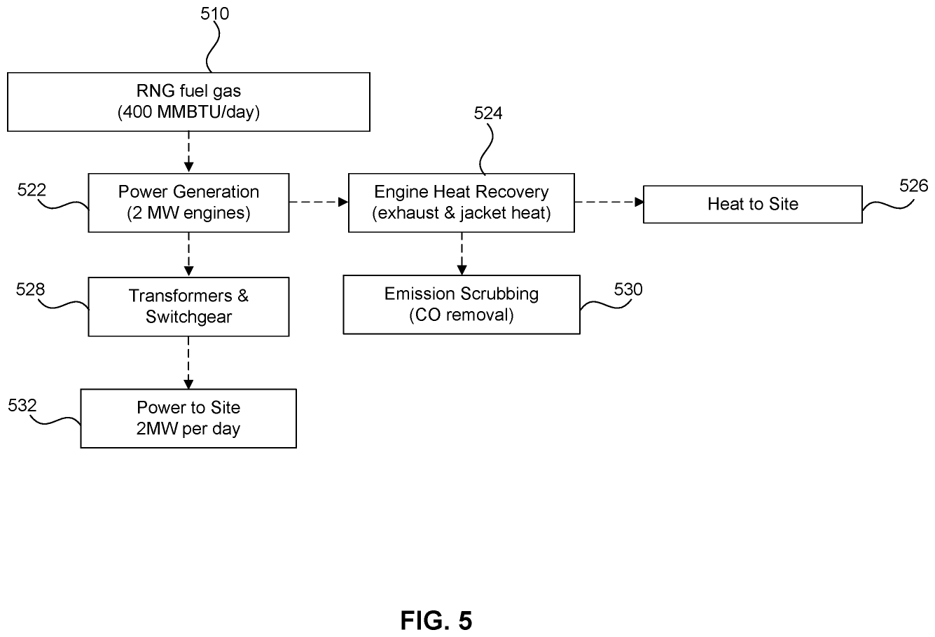

FIG. 5 illustrates an exemplary process for using renewable natural gas 518 to power various processes (e.g., feedstock preparation 100, anaerobic digestion 300, biogas processing system 500, and nutrient recovery 700. For example, renewable natural gas 518 may be transferred to engines 522. Engines 522 and engine heat recovery 524 may together provide heat 526, which can be used to heat various processes. The system may also include transformers and switchgear 528 that provides power 532 to the site. The renewable natural gas 518 may be able to provide at least 1.5 MW per day (e.g., at least 2 MW per day or at least 2.5 MW per day). The renewable natural gas 518 may be able to provide about 1 MW per day to about 3 MW per day (e.g., about 1.5 MW per day to about 2.5 MW per day). The system may also include scrubber 530 to scrub carbon monoxide.

When the CH₄ is used to produce renewable hydrogen at reformer 519, the CH₄ may be passed through a steam-methane-reforming process to react the methane with steam. In some embodiments, the methane is reacted with steam under pressure from about 3 bar to about 25 bar (e.g., from about 5 bar to about 20 bar, from about 10 bar to about 15 bar). In some embodiments, the methane is reacted with steam in the presence of a catalyst.

The system may use one or more scrubbers (e.g., scrubber 506). In some embodiments, scrubber 506 is a 2-stage scrubber with an aerated bioscrubber first stage and a buffered bioscrubber second stage. The aerated bioscrubber that removes NH₃ and/or H₂S can use bacteria belonging to family Thiobacillacaeae (e.g., Thiobacillus sp.) in a sulfur-oxidizing bacterial ecosystem. This produces a low-pH waste slurry that is rich in microbially-derived sulfuric acid. This slurry may be used instead of synthetic acids. The buffered bioscrubber may be used to avoid the loss of methane to the aqueous phase via prolonged residence times in the aerated bioscrubber. The buffered bioscrubber may use bacteria belonging to family Ectothiorhodospiraceae (e.g., Thioalkalivibrio sp.) in halophilic sulfur-oxidizing bacterial ecosystem. This produces an Ectothiorhodospiraceae waste slurry that is rich in bio-sulfur particulate. These sulfur-rich fractions may serve as a microbial source used downstream to produce controlled release fertilizer.

### Nutrient Recovery

Manure digestates (e.g., those produced from poultry litter) are a product of the microbial breakdown of manure under anaerobic conditions. Digestion shares many parallels with fermentation and composting in terms of final products. The composition of the manure feedstock can also have a noticeable effect of the final digestate as manures that are richer in woody; more lignified material will produce digestates that are richer in humic and fulvic acids. Similarly, manures that are richer in nitrogen will often produce digestates characterized by high ammonium and ammonia concentrations. All manure contains both feces and a biomass component. And manure digestion of the biomass is the microorganism-mediated decomposition of cellulose, hemicellulose, and lignin. Lignin is primarily composed of aromatic polyphenolics and while it is relatively degradable in aerobic environments, it is often particularly refractory in anaerobic ones due to the lack of oxygen. However, this benefits the utilization of digestates for fertilizer, because with increased residence time, as the microbes consume the lipids, proteins, and carbohydrates, the lignin-based aromatic macromolecules show a steady increase in concentration. As opposed to composting, anaerobic digestion tends to support the production of more highly functionalized aromatic structures with a higher ion-loading capacity. They often contain a greater proportion of hydroxyl, methoxyl, and carboxyl groups and thus behave as weak polyelectrolytic acids with a large buffering capacity across a wide pH range. These aromatic structures are also known as humic and fulvic acids and are an important contributor to soil buffer and cation exchange capacities. The content of humic substances in composts and digestates is widely used as an indicator of their maturity, safety, and positive impact in soils as an organic amendment or fertilizer.

When manure is field applied, it will often lead to local anaerobic zones due to moisture retention and excess levels of lipids, proteins, and carbohydrates available to the soil microbes. These conditions are ideal for nitrification and denitrification microbes, which in turn generate unwanted emissions from the available nitrogen. At a high level because most of the easily degradable components from the manure are decomposed during anaerobic digestion, there is no metabolizable food remaining for soil microbes. The decomposition also reduces the digestate moisture retention capacity and contributes humic acids, which retard nitrogen mobility. With less moisture, greater buffering capacity, and less decomposable components, field applied digestate rarely causes the same anaerobic conditions that manure does. Further, there may be compounds present in anaerobically digested manure that have a direct depressive effect on soil nitrifiers and denitrifiers, further improving its fertilizer value. Collectively, this leads to digestate having significantly lower gaseous emissions than manure and fewer nitrates because the anaerobic conditions tend to drive available nitrogen to reduced forms like ammonia instead of oxidized forms.

Nutrients may be recovered from the digestate of anaerobically digested animal waste, specifically, poultry litter. As discussed above, the digester may produce a digestate 310 having a solids content of about 3.5% to about 5%. However, in some embodiments, the digestate is removed from the anaerobic digester after a concentrating step. In some embodiments, the concentrating step provides a digestate having greater than 5% solids. In some embodiments, the digestate has from about 5% to about 15% solids, (e.g., from about 5% to about 10% solids).

In some embodiments, solids 706 will be isolated from digestate 310. The solids 706 may be concentrated at forward osmosis concentrate 708. The concentrate 708 may be processed in a mixing tank (e.g., mix tank 734). The pH of concentrate 708 may be reduced to about 2 to about 4. In some embodiments, the pH of concentrate 708 is reduced to less than about 4 (e.g., less than 3) using an acid (e.g., H₂SO₄). The mixture in mix tank 734 may be added to a centrifuge (e.g., centrifuge 736), and the mixture may be separated into sludge paste 737 and centrate 738. Sludge paste 737 may be a humate-rich paste. Centrate 738 is further processed in mix tank 740 by increasing the pH of the centrate 738 to about 7 to about 9. In some embodiments, the pH of centrate 738 is increased to greater than 7 (e.g., greater than 8, greater than 9). The mixture from mix tank 740 may be added to centrifuge 742 and the mixture may be separated into sludge paste 746 and centrate 744. Sludge paste 746 may be a fulvate-rich paste. Both sludge paste 737 and sludge paste 746 may be further processed to produce slow or controlled release fertilizer.

Digester sulfur and nitrogen levels are important for maintaining system stability. High concentrations of nitrogen and sulfur species may be detrimental to anaerobic conditions. This is especially challenging in systems designed to recycle water and in systems that rely on high nitrogen feedstock like poultry litter and meat wastes. In some embodiments, within the reactor, ammonia may be maintained below about 2000 mg/L and Total Kjeldahl Nitrogen (TKN) may be maintained below about 3000 mg/L. In some embodiments, sulfur, as defined by levels in the biogas, should be maintained below about 50,000 mg/L. This may be achieved by targeting maximum production of osmosis and distillation waters from the digestate to be returned to the digesters. In some embodiments, the design objective is to recover water from the digestate that is equable in quality to groundwater specifications. In some embodiments, to the design objective is zero-liquid-discharge (ZLD). Utilizing a zero-liquid-discharge approach the levels of nitrogen and sulfur in the return water can be controlled and maintained indefinitely.

In some embodiments, levels of tannins, humins, and fulvins are increased in their acid or salt forms, and these species are the product of lignin decomposition. Assuming digestion has occurred, which corresponds to about a 50% reduction in COD, then these species can be roughly defined as the Total Organic Carbon (“TOC”) fraction of the Total Carbon (“TC”) present in the Volatile Dissolved Solids (“VDS”) fraction of the produced digestate. In some embodiments, the concentration of combined tannins, humins, and fulvins is in the range of about 7% to about 60% of the total solids in the digestate (e.g., about 7% to about 50% of the total solids in the digestate, about 8% to about 40% of the total solids in the digestate). In some embodiments, the concentration of combined tannins, humins, and fulvins is not less than about 7% of the total solids in the digestate. In some embodiments, the concentration of combined tannins, humins, and fulvins is not less than about 8% of the total solids in the digestate. In some embodiments, the concentration of combined tannins, humins, and fulvins in the digestate is in the range of about 1,500 mg/L to about 40,000 mg/L (e.g., about 10,000 mg/L to about 30,000 mg/L or about 15,000 mg/L to about 25,000 mg/L). In some embodiments, the concentration of combined tannins, humins, and fulvins in the digestate is at least about 5,000 mg/L, at least about 10,000 mg/L, at least about 15,000 mg/L, or at least about 20,000 mg/L, at least about 25,000 mg/L, at least about 30,000 mg/L, at least about 35,000 mg/L, or at least about 40,000 mg/L. This level of tannins, humins, and fulvins supports recovery of all nutrients and the production of controlled and slow-release fertilizers from the digestate.

The terms “tannin”, “tannins”, “tannic acid”, or “tannic acids” are used herein to mean any large polyphenolic compound containing sufficient hydroxyls and other suitable groups (such as carboxyls) to form strong complexes with various macromolecules. The terms “humin”, “humins”, “humic acid”, or “humic acids” are used herein to mean any large polyphenolic compound containing sufficient hydroxyls and other suitable groups (such as carboxyls) to form strong complexes with various macromolecules. Tannins have molecular weights ranging from about 500 to over about 3,000 (gallic acid esters) and up to about 20,000 (proanthocyanidins). Hydrolyzable tannins are composed of gallic acid subunits, phlorotannins are composed of phloroglucinol subunits, and condensed tannins are largely flavone derived. A typical humic acid will have a similar molecular weight range from about 1,000 to about 20,000 u, but a broader variety of components including quinone, phenol, catechol, and sugar moieties. A typical humic substance is a mixture of many molecules, most of which are based on a motif of aromatic nuclei with phenolic and carboxylic substituents (polyphenols). The biggest difference between tannins and humins is based on mechanism of formation. Tannins are produced by plants, but humins are a degradation and decomposition product. The terms “fulvin”, fulvins”, “fulvic acid”, or fulvic acids” are used herein to mean any large polyphenolic compound containing sufficient hydroxyls and other suitable groups (such as carboxyls) to form strong complexes with various macromolecules. The defining aspect of fulvic acids is that they are typically smaller versions of the bulkier polymers (tannic/humic). In general, the differences are related to the carbon and oxygen contents, acidity, degree of polymerization, molecular weight, and color.

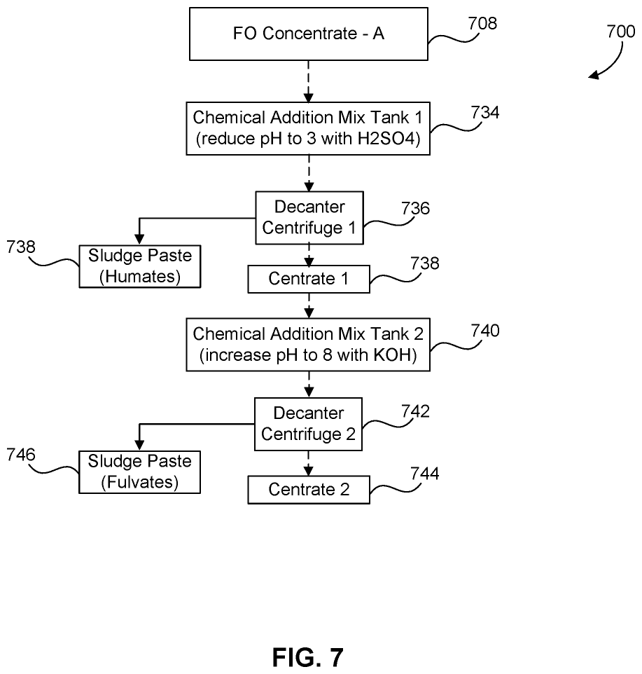

In some embodiments, the tannins, humins, and fulvins are separately recovered from the digestate using various acid and base driven precipitations (FIG. 7). Each of these organic species have established market value independently and in combined forms. For example, if the pH is lowered to about 3-4 using sulfuric acid then the humins will precipitate but the fulvins will stay in solution. If the humins are removed and the pH is then elevated to about 8-9, the fulvins precipitate. In some embodiments, after the humins and fulvins are removed, 80% to 90% of the Total Organic Carbon (TOC) fraction of the Total Carbon (TC) present in the Volatile Dissolved Solids (VDS) fraction will have been removed.

In the process of recovering and recycling the maximum amount clean water from dirty water, target fractions may be removed in series to allow increasing levels of filtration until it is no longer practical to recover any additional water. This approach can be re-applied to anaerobic digestion as a method of progressively fractionating the non-degradable organic fractions and inorganic fractions towards the production of value-added fertilizer products. In some embodiments, the clean water that is produced can be recycled to the anaerobic digester to reduce environmental footprint and improve sustainability. In some embodiments, the final stage and treatment range in all systems may be reverse osmosis in addition to evaporation and distillation to ensure recovery of nutrients.

In some embodiments, sulfuric acid, phosphoric acid, lactic acid, nitric acid, acetic acid, lactic acid, peracetic acid, oxalic acid, citric acid, and/or slurries containing high levels of one or more of these acids independently or in combination with oxidants such as peroxides, permanganates, and hypochlorites are used to treat liquid/solid anaerobic digester outputs (digestates) rich in tannins, humins, and fulvins in their acid or salt forms to fully solubilize any bound phosphorous and improve its availability for further reactions. These reaction kinetics improve at elevated temperatures up to about 100° C.

In some embodiments, slurries containing high levels of acids may be used. These slurries may be the microbial sulfur-rich streams from scrubber 506 discussed above. In some embodiments, the microbial sulfur-rich streams (e.g., the low-pH slurry rich in bio-sulfuric acid) may be used directly without further processing in the production of controlled release fertilizers. Other microbial acid slurries may be used. For example, microbial citric acid, acetic acid, lactic acid, sulfuric acid, or combinations thereof may be used. Using waste streams from the scrubber 506 has several benefits, including reducing waste, reducing costs related to sourcing input materials or waste handling, and increasing use of organic materials in the process.

Adding oxidants may oxidize functional groups on the humic acids to carbonyls and aldehydes. This may reduce the charge capacity of humic acid, which can lead to coagulation. Oxidants can be too expensive to use extensively; however, the inventors have unexpectedly found that oxidants may be used in conjunction with an acid to increase the concentration of associated phosphates and potassium in addition to improving the coagulation effect. Any combination of the acids and oxidants listed above may be used.

Digestates that are rich in ammonia and ammonium species may present a challenge related to emissions and leaching. The most practical way to improve manure digestate performance while reducing environmental impacts is to convert it into a controlled release form. The Association of American Plant Food Control Officials (AAPFCO) defines controlled release fertilizers as a fertilizer containing a plant nutrient in a form which delays its availability for plant uptake and use after application, or which extends its availability to the plant significantly longer than a reference rapidly available nutrient fertilizer like ammonium nitrate or potassium nitrate. For example, a non-controlled release fertilizer will release 100% of its nitrogen content in less than 24 hours, while a controlled release fertilizer will take longer than 24 hours to release all of its nitrogen content. The European Union also has established standards for controlled release fertilizers in BS EN 13266-2001 and BS ISO 18644-2016. These standards define controlled release as a fertilizer that releases its nutrient or nutrients under defined conditions. This a function of the standardization expected with controlled release fertilizers since the factors dominating the rate, pattern, and duration of nutrient release are known and predictable with these products.

Due to the nutrient intensity of manure digestates being lower than rapidly available fertilizers like urea and nitrates and due to the generally distributed production of manure digestates at either farm scale or sub-regional scale, one controlled release methodology is matrix based, also sometimes referred to as controlled-loss, or loss-control fertilizers. In some embodiments, matrix based fertilizers may use the addition of various silicate compounds to improve the controlled-release characteristics of the fertilizer. In some embodiments, silicates are present in an amount by weight of about 1% to about 20% by weight (e.g., about 2% to about 18%, about 3% to about 16%, about 4% to about 14%, about 5% to about 12%, about 6% to about 10%). In some embodiments, the silicates are present in an amount by weight of about 2.5% to about 20%.

A major class of silicates are the aluminosilicates, which are frequently encountered as clays, feldspars, and zeolites. Clays and zeolites have important differences. Clays are phyllosilicates or hydrated layered silicates that form parallel sheets of silicate tetrahedra. This gives them plasticity and allows them to expand and trap water and ions very efficiently. Clay minerals are able to exchange ions and intercalate neutral molecular species between the interlayer regions by interaction with structural water. The faces of each platelet also possess a net negative charge, which provides the ability to directly associate with cations. Zeolites are tectosilicates or framework silicates and have a three-dimensional framework of silicate tetrahedra. This makes them rigid and microporous and gives them a high density of binding sites, particularly for cations. Their framework also forms paths capable of promoting ion exchange, adsorption, selective diffusion and component separation depending on molecule size and shape. Generally, clays are mined from natural deposits, but zeolites can be both synthetically produced as well as mined, depending on the target market.

In some embodiments, the fertilizer matrix contains aluminosilicates like clays and zeolites. In some embodiments, the fertilizer matrix controlled release performance occurs via nutrient adsorption and diffusion control. In some embodiments, the nutrient adsorption and diffusion control effects are frequently witnessed at the addition of about 2.5% to about 1020% by mass of clays and zeolites. As a result, only a small amount of clay or zeolite needs to be blended into the fertilizer matrix to see a noticeable effect. Both have well established effects on nitrogen loss due to their three-dimensional lattice structure and combination of microchannel and nano-channel networks. This architecture retards nitrogen release via surface tension, viscous forces, hydrogen bonding, and steric hindrance. As a function of being aluminosilicates, they also contain a significant level of negative surface charge density, which allows them to interact broadly with positively charged cationic species like ammonium. This is similar in some ways to humic and fulvic acids which can achieve comparable performance via carboxyl and hydroxyl interactions. In some embodiments, clays and zeolites are blended with humic-acid digestates and/or fulvic-acid rich digestates, resulting is a highly favorable matrix for controlled nutrient release.

In some embodiments, the proposed method generates a matrix-based controlled release fertilizer digestate product, meeting the performance criteria outlined in the BS EN 13266-2001 and BS ISO 18644-2016 standards.

Methods disclosed herein meet these criteria through primarily matrix-induced effects. But these methods may also include mechanisms for binding the ammonium species in various slow-release forms as needed. In some embodiments, the addition of about 5 to about 20% by sludge mass (dry basis) clay or zeolite helps to achieve the desired controlled release fertilizer performance criteria. Less clay and/or zeolite will be needed if the nitrogen is bound up in a slow release form as opposed to an ammonium form. The primary method for achieving controlled release is through controlled water solubility either through coatings/encapsulation or matrix-enhancement with materials like sulfur, clay, zeolites, humates, fulvates, tannanates, polymers, natural nitrogenous organics, or protein materials. Given that some of these materials are also recommended as sludge bulking agents for improving centrifuge performance, selection of the appropriate material is likely to fill multiple roles (i.e. centrifuge optimization and fertilizer performance). In some embodiments, the clay and zeolite species that will be used include one or more of the following:

Halloysite—Al2Si₂O₅(OH)₄;

Kaolinite—Al₂(OH)₄Si₂O₅;

Illite—(K,H₃O)(Al,Mg,Fe)₂(Si,Al)₄O₁₀[(OH)₂,(H₂O)];

Montmorillonite—(Na,Ca)_{0.33}(Al,Mg)₂Si₄O₁₀(OH)₂·nH₂O;

Talc—Mg₃Si₄O₁₀(OH)₂;

Sepiolite—Mg₄Si₆O₁₅(OH)₂·6H₂O;

Pyrophyllite—Al₂Si₄O₁₀(OH)₂;

Erionite—(Na₂,K₂,Ca)₂Al₄Si₁₄O₃₆·15H₂O;

Stilbite—NaCa₂Al₅Si₁₃O₃₆·17H₂O; and

Mordenite—(Ca,Na₂,K₂)Al₂Si₁₀O₂₄·7H₂O.

In some embodiments, the method includes a process to convert a portion or all of the mobile ammonium species into bulkier, slow-release forms. In some embodiments, the digestate may contain very high levels of ammonium carbonate (NH₄CO₃) (e.g., about 1000 mg/L to about 3000 mg/L). This ammonium may remain in the NH₄ form if it is associated with a stronger acid and if the temperature of the water does not exceed about 80° C. However, at elevated temperatures, ammonium carbonate will decompose into ammonia (NH₃) and carbon dioxide (CO₂) gas. Similarly, if the pH is increased using a calcium or magnesium base, these species will replace the NH₄ and associate with the carbonate (CO₃) instead. When this occurs, the NH₄ is converted into NH₃. In some embodiments, ammonium will be converted in-situ into hexamine and/or ammonium-aldehyde adducts using an aldehyde, for example, formaldehyde, acetaldehyde, furfural, isobutyraldehyde, butyraldehyde, or propionaldehyde. This conversion is a derivative of a commercially practiced reaction with urea that is used for generating some of the more widely available slow-release nitrogen fertilizers (urea-formaldehyde, urea-isobutyraldehyde/isobutylidene diurea, urea-acetaldehyde/cyclo diurea, etc.). In some embodiments, ammonium carbonate will be converted in-situ into hexamine using formaldehyde. This conversion proceeds quite rapidly under normal process conditions. In some embodiments, the hexamine will be associated with tannic, humic, or fulvic acids. Controlling the ammonium in this way avoids issues related to NH₃ emissions and toxicity and has additional benefits related to crystallizer operations downstream. In some embodiments, the controlled release fertilizer reduces nitrogen emissions by at least 10% (e.g., at least 15%, at least 20%, or at least 25%). In some embodiments, the controlled release fertilizer reduces nitrogen emissions by about 10% to about 40% (e.g., about 15% to about 30% or about 20% to about 25%).

Another benefit of adding formaldehyde and/or other aldehydes to a solution rich in ammonium to form hexamine and other high molecular weight organic ammonium adducts is the “bulking” factor of such addition. When formaldehyde and/or other aldehydes are added to form hexamine and other high molecular weight organic ammonium adducts, the size of the compounds increases such that they can be captured by a nanofilter. Nanofilters are capable of rejecting bulky divalent ions like those formed by calcium and magnesium, but ammonium and monovalent ions like potassium and sodium are too small and will pass through. For example, the hydrated cationic radius of calcium chloride and magnesium chloride is 9.6 A and 10.8 A respectively. These can be rejected by a nanofilter with a membrane aperture of 1 nm to 2 nm. In contrast, the hydrated radius of ammonium chloride and sodium chloride is 5.3 A and 5.6 A respectively, which is too small for effective rejection by a nanofilter. But non-hydrated hexamine crystals form a cubic lattice whose edge is 7.02 A, and hydrated hexamine crystals have a larger radius—similar to those observed by calcium chloride and magnesium chloride—that can be rejected by a nanofilter.

This increase in size is important for a zero-liquid discharge system. For example, the zero-liquid discharge process may include a series of filters, including nanofilters. Nanofilters have high flux exhibited by filters (e.g., ultrafiltration filters) while also rejecting ions like membranes (e.g., reverse osmosis membranes and forward osmosis membranes). This means that the in-situ conversion of ammonium to hexamine or similar adducts will support the rejection of these nitrogen species from the clean permeate (e.g., reverse osmosis permeate, discussed below). Compared to other methods, rejection of ammonium across a nanofilter provides a simple and economical way to remove ammonium.

In some embodiments, ammonium is controlled using lignin amination, for example humic/fulvic amination which includes: producing Organosolv lignins; repeating lignin Mannich reactions; characterizing the products (e.g. FTIR, CHN, MS, UV-Vis, and NMR); using this method as basis for amination protocol with hexamine and/or NH₄ carbonates in AD digestate; repeating the process on humic, fulvic, and tannic standards; and repeating the process on AD digestates.

In some embodiments, ammonium is controlled using ammonium aldehyde reactions followed by precipitation mechanisms which includes: producing hexamine and similar adducts from ammonium carbonate & bicarbonate and formaldehyde; replicating procedure with multiple aldehydes (e.g. formaldehyde, acetaldehyde, furfural, isobutyraldehyde, butyraldehyde, or propionaldehyde acetaldehyde, isobutylaldehyde); characterizing the products (e.g. FTIR, CHN, GC, and NMR); using adhesive chemical protocols/mechanisms for guidance, test effects of phenolic additions on hexamine precipitation and/or molecular product formation (both base and acid catalyzed). Target phenolics are phenol, resorcinol, cresol, and phloroglucinol; establishing how mechanisms can be repeated using AD digestates; exploring effects of other process variables such as pH, halogen salts, clay & zeolite addition, and chlorine salts.

In some embodiments, oxidants such as peroxides, permanganates, and hypochlorites acids independently or in combination with sulfuric acid, phosphoric acid, nitric acid, acetic acid, peracetic acid, oxalic acid, citric acid, and/or slurries containing high levels of one or more of these oxidants and acids are used to treat liquid/solid anaerobic digester outputs (digestates) rich in tannins, humins, and fulvins in their acid or salt forms to convert them into forms with a higher concentration of carbonyl groups in support of in-situ Mannich-like aminations via ammonium and aldehyde reaction mechanisms/pathways. This to be followed by addition of reactive phenolics such as phenol, resorcinol, cresol, and/or phloroglucinol to initiate polymerization reactions contributing to increased molecular weight and improved settling/recovery of the organic nitrogen.

Another benefit to adding reactive phenolics listed above is precipitating hexamine from solution (e.g., wastewater). Hexamine can contaminate drinking water, and it is difficult to remove from wastewater. But hexamine may behave like a resin component and react effectively with reactive phenolics, such as those listed above. When it reacts, the hexamine will precipitate, allowing for easy removal from the solution.

In some embodiments, the method includes a drying process. In some embodiments, the water of crystallization provides struvite as a hexahydrate and cement as a decahydrate. Crystallization of hydrates is an important principal for producing a dry product with minimal evaporation. In some embodiments, avoiding an evaporation process that includes heating will prevent unwanted thermal decomposition under dryer conditions.

Ammonium carbonate may decompose to NH₃ gas and CO₂ at around 80° C. By adding sulfuric acid, phosphoric acid, nitric acid, acetic acid, lactic acid, peracetic acid, oxalic acid, citric acid, and/or slurries containing high levels of one or more of these acids before a dryer, the NH₄ may be prevented from going into the vapor phase as NH₃ during drying. In some embodiments, sulfuric acid or a slurry containing high levels of sulfuric acid is added before a dryer. In some embodiments, acids made by natural processes (e.g., acetic acid, citric acid, lactic acid, and sulfuric acid) are used. Fertilizers made using acids made by natural processes may be organic fertilizers. The other dryer benefit is that sulfate salts are hydrous and go into the solid phase at lower temperatures and with less evaporation than is necessary for chloride salts, which lowers the energy demand. However, low pH process water cannot be fed into the dryer due to corrosion issues. In some embodiments, sulfuric acid will be added to control NH₄. Further, the process water may be buffered back up to about pH 8-9 using a buffer. It has been unexpectedly found that, unlike divalent salts, monovalent salts do not disrupt the sulfate association, which can reduce the loss of NH₃ as a vapor.

In some embodiments, other chemical addition will be utilized to further lower energy demand, reduce capital expenditures, and improve properties after drying. In some embodiments, the chemical additions include at least one of zinc sulfate; zinc oxide; ferrous sulfate; iron(III) oxide-hydroxide; aluminum hydroxide; silica fume/ground silica/ground rice hull; dolomite; and metakaolin/various pozzolans/cement kiln dust.

The silica fume, dolomite, and metakaolin are well-regarded alkali cement reagents and will react with the concentrated slurries upstream of the dryer to create a gel. Typically, these are used in low concentrations, but the specific amounts may depend on the amount of water in the digestate. These reagents may be added to the slurry before it entered the dryer. In some embodiments, an addition of about 1% by mass of silica fume, dolomite, and metakaolin to improve properties after drying. In some embodiments, metakaolin is added at about 1% by mass and mixed well. In some embodiments, about 1% by mass of silica fume/ground silica is add to the metakaolin mixture. In some embodiments, about 1% by mass of dolomite to the metakaolin-silica mixture. In some embodiments, the number/sequence of additions of silica fume, dolomite, and metakaolin is determined by the properties of the slurry before drying (i.e. viscosity, solids concentration, pH, etc.).

The zinc sulfate/zinc oxide and the ferrous sulfate/iron hydroxide and aluminum hydroxide support an entirely different mechanism related to the minerals jarosite and alunite, and ammonium zinc sulfate hydrate. These salts are capable of both binding ammonium/potassium and making the resultant crystals even more hydrous. In some embodiments the more hydrous the solids are, the lower the drying process intensity will be. In some embodiments, zinc sulfate/zinc oxide and ferrous sulfate/iron hydroxide and aluminum hydroxide are added to the slurry as its being concentrated in a dryer and can initiate crystallization earlier and support the development of noticeably different crystals. For example, the crystals may be larger sizes, which is indicative of favorable thermodynamics. In digestate, these salts can also be potent coagulants for the dissolved organics (humic, fulvic, and & tannic acids). By virtue of the metallic character and ionic radius of zinc sulfate/zinc oxide and ferrous sulfate/iron hydroxide and aluminum hydroxide, they have proven to be effective at creating solids with less energy input. In some embodiments, about 0.5% to about 1.5% by mass of zinc sulfate/zinc oxide and ferrous sulfate/iron hydroxide and aluminum hydroxide is added to provide solids with less energy input. In some embodiments, about 1.0% to about 1.5% by mass of zinc sulfate/zinc oxide and ferrous sulfate/iron hydroxide and aluminum hydroxide is added to provide solids with less energy input. In some embodiments, about 0.5% by mass of zinc sulfate/zinc oxide and ferrous sulfate/iron hydroxide and aluminum hydroxide is added to provide solids with less energy input. Similar to the hexamine method containing processes to bind ammonium with organic chemistries for slow-release, aspects of the objectives of this research are an important part of the overall proposed method because they present a pathway towards ensuring that the digestate meets controlled release specifications in the event that a matrix-based approach proved insufficient.

As part of the nutrient recovery 700, the system may include various processes for water treatment such that the system achieves zero-liquid discharge. For example, the system may include various filters, reverse osmosis systems, and forward osmosis systems. In some embodiments, the system may include spiralwater filter 702 that produces solids 706 and filtrate 704. Draw solution 710 may be used with both forward osmosis membranes 709 and reverse osmosis membranes 712. Reverse osmosis permeate 714 (e.g., water) from reverse osmosis membranes 712 may be moved to water storage 200. Centrate 744 may be passed through forward osmosis membranes 718. Draw solution 720 may be used with both forward osmosis membranes 718 and reverse osmosis membranes 730. Reverse osmosis permeate 732 (e.g., water) from reverse osmosis membranes 730 may be moved to water storage 200. Forward osmosis concentrate 722 may be passed through evaporator 724 to produce brine solids 726 and distilled water 728. Brine solids 726 may be polyhalite brine solids. Distilled water 728 may be moved to water storage 200. As discussed above, water storage 200 may be used to provide water to feedstock preparation 100.

### Emissions Reduction

The processes disclosed herein may reduce emissions such that the process produces emissions offset credits (e.g., carbon credits, carbon offsets, etc.). Some emissions offset credits, such as renewable energy certificates (“RECs”), offset kilowatt-hours instead of carbon. The emissions offset credits help reduce greenhouse gas emissions. The emissions offset credits may be a tradeable certificate that gives the owner a right (e.g., a permit) to emit a set amount of carbon dioxide or the equivalent amount of a different greenhouse gas (e.g., NO₂) or a set amount of kWh. For example, the credits may give the owner a right to emit one ton per year of carbon dioxide or the equivalent amount of a different greenhouse gas.

Emissions offset credits are measured in tons of carbon dioxide equivalent (“CO2e”) or kilowatt-hour (kWh), and the emissions offset credits may be traded on both private and public markets. Prices of credits are primarily driven by supply and demand in the markets. Accordingly, prices can fluctuate based on supply and demand domestically and internationally. Examples of exchanges that specialize in the trading of the credits include the European Climate Exchange, the NASDAQ OMX Commodities Europe exchange, and the European Energy Exchange.

In some embodiments, the processes disclosed herein may reduce emissions sufficient to produce at least about 250,000 tons per year of CO2e offset (e.g., at least about 500,000 tons per year of CO2e offset, at least about 750,000 tons per year of CO2e offset, or at least about 1,000,000 tons per year of CO2e offset. In some embodiments, the processes disclosed herein may reduce emissions sufficient to produce about 250,000 tons per year to about 1,500,000 tons per year of CO2e offset (e.g., about 500,000 tons per year to about 1,000,000 tons per year of CO2e offset, or about 500,000 tons per year to about 750,000 tons per year of CO2e offset). In some embodiments, the processes disclosed herein may reduce emissions sufficient to produce about 550,000 tons per year of CO2e offset per year. In some embodiments, the CO2e offset per year is achieved due to reduction in nitrogen (e.g., NO₂) emissions in the processes disclosed herein.

### EXAMPLES

### Example 1

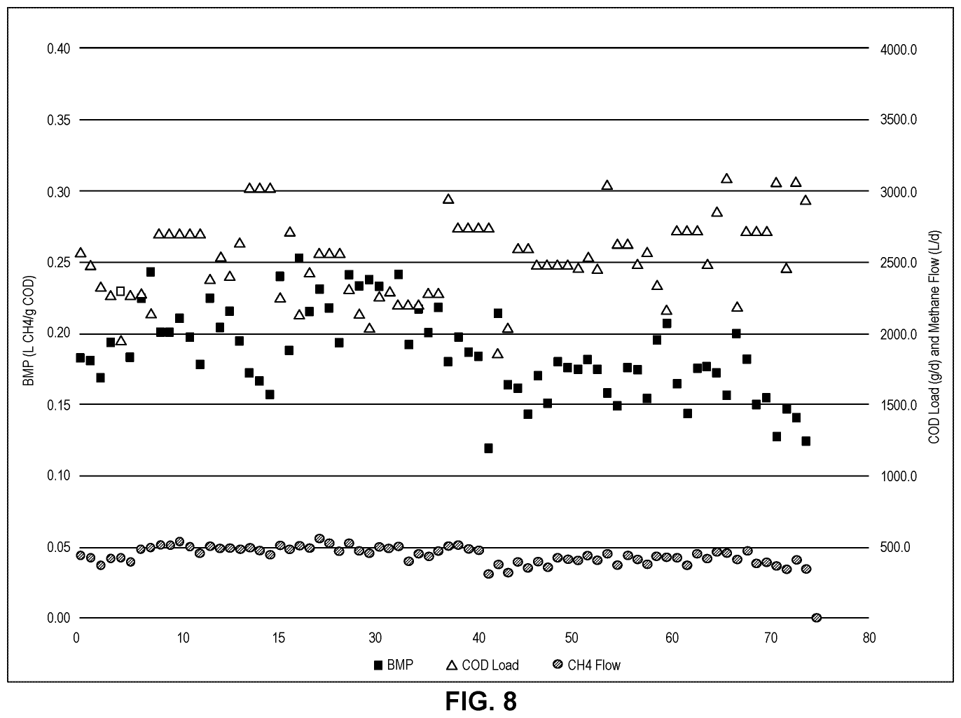

In one experiment, the predictability, stability, and effectiveness of methods according to the embodiments disclosed herein was evaluated. This was done by measuring the biochemical methane potential (“BMP”), COD load, and methane flow. FIG. 8 shows the trends of these values over the course of a representative 80 days.

As shown by FIG. 8, it is possible to make enough COD from poultry litter in a predictable and stable system while also generating a significant amount of methane (about 400 L per day to about 550 L per day).

### Example 2

In one experiment, a conical refiner (e.g., conical refiner 114) was used just before digestion. Samples were tested at different distances the rotating grinder (i.e., a rotor) and the stationary cutter (i.e., a stator). During run 2, the distance between the rotor and stator was 3 times the distance for run 1. Table 1 below shows the dilution factor and COD for run 1. Table 2 shows the dilution factor and COD for run 2.

TABLE 1

| | Sample 1 | Sample 2 | Sample 3 |
|---|---|---|---|
| Dilution factor | 103 | 125 | 96 |
| COD (mg/L) | 91,600 | 91,000 | 89,300 |

TABLE 2

| | Sample 1 | Sample 2 | Sample 3 |
|---|---|---|---|
| Dilution factor | 104 | 100 | 104 |
| COD (mg/L) | 67,300 | 68,800 | 73,000 |

The average COD for run 1 was 90,600 and the average COD for run 2 was 69,700. Accordingly, this experiment shows that the COD may be affected based on the distance between the rotor and stator. As shown by this example, it is possible to generate enough COD from poultry litter to generate a controlled release fertilizer and biogas.

### Example 3

In one experiment, various fertilizer samples were tested for controlled release properties. Samples A-C were produced using methods according to the embodiments described herein. Sample D was a commercially available fertilizer sample. A carbon, hydrogen, and nitrogen analysis was performed on each sample before testing using an Elementar VarioMacroCube. Each sample included the compositions shown in Table 3 below.

TABLE 3

| Sample | Carbon (%) | Hydrogen (%) | Nitrogen (%) |
|---|---|---|---|
| A | 13.22 | 3.38 | 7.31 |
| B | 15.38 | 3.38 | 6.89 |
| C | 15.06 | 3.26 | 5.88 |
| D | 36.41 | 5.57 | 9.13 |

Each sample's controlled release characteristics were tested by dissolving the sample in deionized water at 1% w/w and stored in a sealed container at room temperature. Each sample was stirred periodically over 24 hours. After 24 hours, the liquid was decanted and the remaining solid was dried at room temperature for 48 hours. After 48 hours, the samples were weighed. A carbon, hydrogen, and nitrogen analysis was again performed on all samples to determine elemental composition after the experiment. The change in elemental composition of each sample is shown in Table 4 below.

TABLE 4

| | Change in | Change in | Change in | Change in |
|---|---|---|---|---|
| Sample | mass (%) | Carbon (%) | Hydrogen (%) | Nitrogen (%) |
| A | −60.5 | −7.85 | −48.45 | −76.11 |
| B | −57.9 | −11.39 | −43.55 | −71.58 |
| C | −54.6 | −10.56 | −39.63 | −64.29 |
| D | −44.3 | −34.22 | −32.14 | −35.64 |

A non-controlled release fertilizer would release 100% of its nitrogen in less than 24 hours. As shown in Table 4, after 24 hours Samples A-C each retained significant portions of carbon, hydrogen, and nitrogen, meaning the samples behaved as a controlled release fertilizer.

### Example 4

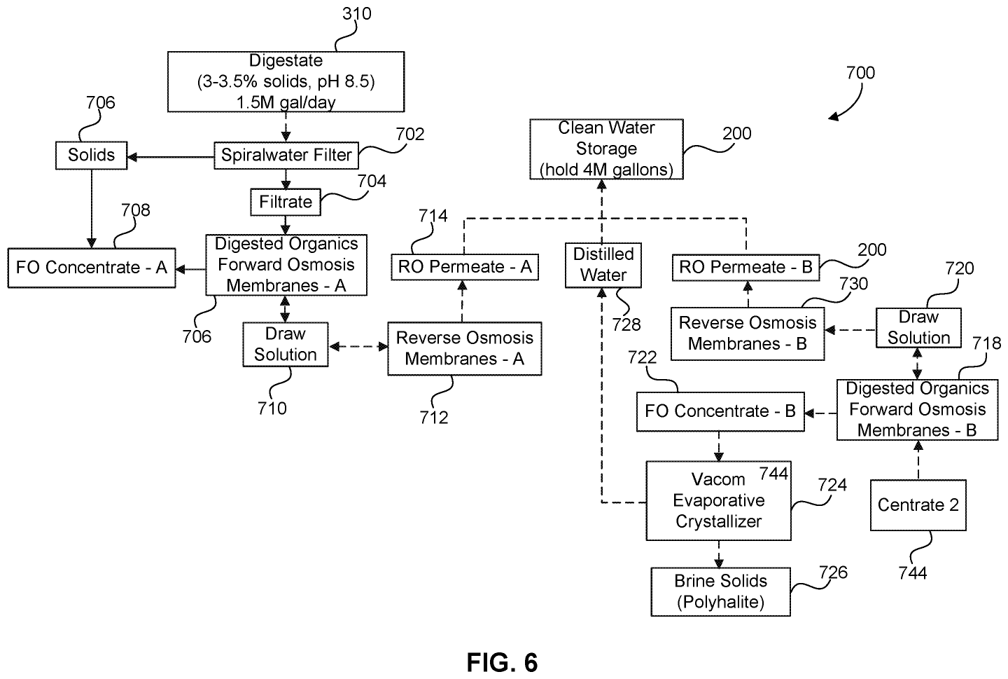

As described above, the methods disclosed herein may produce clean water as a byproduct of the processes. For example, reverse osmosis permeate 714 shown in FIG. 6 may be clean water that is moved to water storage 200. In one experiment, the cleanliness of the water produced by the methods disclosed herein was determined by measuring the turbidity and conductivity of the stream.

Turbidity is the measure of relative clarity of a liquid. Turbidity is reported in nephelometric turbidity units (NTU) and is measured by shining a light through the water. Turbidity and clarity are inversely related (e.g., lower turbidity corresponds to more clarity). Generally water at 4 NTU and above will be visibly cloudy, while “crystal-clear” water has a turbidity of <1 NTU.

Conductivity is a measure of the ability of water to pass an electrical current. Conductivity is reported in millisiemens per centimeter (mS/cm). Conductivity in water is affected by the presence of inorganic dissolved solids such as chloride, nitrate, sulfate, and phosphate anions (ions that carry a negative charge) or sodium, magnesium, calcium, iron, and aluminum cations (ions that carry a positive charge). Distilled water has a conductivity in the range of 0.5 to 3 μS/cm (0.0005 mS/cm to 0.003 mS/cm). The conductivity of rivers in the United States generally ranges from 0.05 mS to 1.5 mS/cm. Industrial waters can range as high as 10 mS/cm.

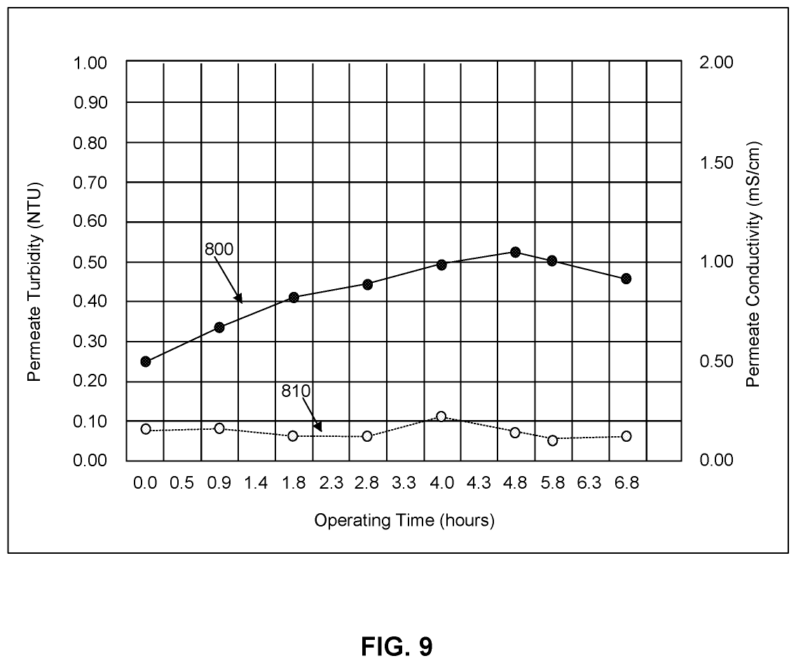

FIG. 9 shows conductivity 800 and turbidity 810 of permeate (e.g., reverse osmosis permeate 714) produced using methods disclosed herein. As shown by line 800, permeate conductivity ranges from about 0.25 mS/cm to about 1 mS/cm over nearly 7 hours of operation. And as shown by line 810, permeate turbidity is about 0.10 NTU or less over nearly 7 hours of operation. Thus, the methods disclosed herein are capable of producing clean water as a byproduct.

It is to be appreciated that the Detailed Description section, and not the Summary and Abstract sections, is intended to be used to interpret the claims. The Summary and Abstract sections may set forth one or more but not all exemplary embodiments of the present disclosure as contemplated by the inventor(s), and thus, are not intended to limit the present disclosure and the appended claims in any way.

The present disclosure has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the disclosure that others can, by applying knowledge within the skill of the art, readily modify and/or adapt for various applications such specific embodiments, without undue experimentation, without departing from the general concept of the present disclosure. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The above examples are illustrative, but not limiting, of the present disclosure. Other suitable modifications and adaptations of the variety of conditions and parameters normally encountered in the field, and which would be apparent to those skilled in the art, are within the spirit and scope of the disclosure.

References in the specification to “one embodiment,” “an embodiment,” “an example embodiment,” “some embodiments,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

The breadth and scope of the present disclosure should not be limited by any of the above-described exemplary embodiments, but should be defined only in accordance with the following claims and their equivalents.

1.-38. (canceled)

39. A process for forming a controlled release fertilizer, the process comprising:
providing a feedstock comprising poultry waste, the poultry waste comprising lignin, the feedstock having a moisture content of about 35% or less;
diluting the feedstock to form a slurry having a moisture content of at least about 80%;
mechanically refining the slurry such that the slurry has an average particle size of about 100 μm or less;
anaerobically digesting the slurry to produce a digestate having a solids content of less than about 10% percent by weight and a biogas, the digestate having a combined concentration of humins, fulvins, and tannis that is greater than a combined concentration of humins, fulvins, and tannins in the feedstock; and
processing the digestate to form a controlled release fertilizer.

40. The process of claim 39, wherein the feedstock comprises greater than about 80% poultry waste.

41. The process of claim 39, wherein the feedstock comprises animal bedding.

42. The process of claim 39, wherein mechanically refining the slurry exposes and releases carbon, polyphenolics, lignin, cellulose, and hemicellulose in the poultry waste.

43. The process of claim 39, wherein anaerobically digesting the slurry converts cellulose and hemicellulose in the poultry waste to biogas.

44. The process of claim 39, wherein the combined concentration of humins, fulvins, and tannins in the digestate is at least about 10,000 mg/L.

45. The process of claim 39, wherein the feedstock has a moisture content of about 25%.

46. The process of claim 39, wherein the slurry has a moisture content of at least about 94%.

47. The process of claim 39, wherein the digestate has a solids content of about 2% to about 5%.

48. The process of claim 39, wherein the slurry has an average particle size of about 10 μm or less.

49. The process of claim 39, further comprising removing carbon dioxide from the biogas to produce a renewable natural gas having a methane concentration of at least about 70% by volume.

50. The process of claim 39, wherein the renewable natural gas has a methane concentration of at least about 90%.

51. The process of claim 39, wherein the mechanical refining is done before the anaerobic digestion.

52. The process of claim 39, wherein the mechanical refining is done using at least one of a conical refiner or a disc refiner.

53. The process of claim 39, wherein the feedstock comprises greater than about 90% poultry waste.

54. The process of claim 39, further comprising removing impurities from the biogas, wherein the impurities comprise at least one of ammonia or hydrogen sulfide.

55. The process of claim 54, wherein the impurities are removed before the carbon dioxide is removed.

56. The process of claim 39, wherein the renewable natural gas is produced at a rate of at least about 3 MMBTU per ton of the feedstock.

57. The process of claim 56, wherein renewable natural gas is produced at a rate in a range of about 3 MMBTU per ton of the feedstock to about 5 MMBTU per ton of the feedstock.

58. The process of claim 39, further comprising:
reducing nitrogen emissions by applying the controlled release fertilizer as a soil amendment.

59. The process of claim 39, further comprising:
processing the biogas to produce an acid-rich stream; and
wherein the processing the digestate comprises mixing the digestate with the acid-rich stream to produce the controlled release fertilizer.

60. The process of claim 39, wherein the biogas comprises methane, and wherein the process further comprises reacting at least a portion of the methane with steam at a pressure from about 3 bar to about 15 bar to produce hydrogen.

61. The process of claim 39, wherein processing the digestate comprises separating water from the digestate for recycle.

62. The process of claim 39, wherein the process reduces nitrogen emissions sufficient to offset from about 1.71 tons to about 10.27 tons of CO₂ equivalent per ton of the feedstock.

63. A process of preparing a controlled release fertilizer composition comprising:
anaerobically digesting poultry litter to produce a digestate having a solids fraction and a liquid fraction, the digestate comprising at least about 10,000 mg/L of combined tannins, humins and fulvins; and
processing the digestate to form a controlled release fertilizer.

64. The process of claim 63, further comprises adding an acid to the solids fraction to produce a humates sludge paste.

65. The process of claim 64, wherein the acid comprises at least one of sulfuric acid or citric acid.

66. The process of claim 64, wherein adding the acid reduces the pH of the solids fraction such that the solids fraction is acidic.

67. The process of claim 64, further comprises adding a base to the solids fraction to produce a fulvates-rich sludge paste.

68. The process of claim 67, wherein the base is potassium hydroxide.

69. The process of claim 67, wherein adding the base increases the pH to about 7 to about 9.

70. The process of claim 63, further comprising treating the liquid fraction to produce polyhalite brine solids.

71. A controlled release fertilizer composition comprising tannins, humins, fulvins, and brine solids,
wherein the controlled release fertilizer is derived from a poultry litter digestate, and
wherein the combined composition of tannins, humins, and fulvins is at least about 10,000 mg/L.

72. The composition of claim 71, further comprising one or more silicates, wherein the one or more silicates comprise clay and zeolite species.

73. The composition of claim 72, wherein the composition comprises about 1% by weight to about 20% by weight silicates, and wherein the clay and zeolite species comprise halloysite, kaolinite, illite, montmorillonite, talc, sepiolite, pyrophyllite, erionite, stilbite, mordenite or combinations thereof.

74. A method of reducing nitrogen emissions, the method comprising applying the composition of claim 71 as a soil amendment.

75. The method of claim 74, wherein nitrogen emissions are reduced by at least about 10%.

76. The composition of claim 71, wherein the brine solids are polyhalite brine solids.
